(19) United States
(12) Patent Application Publication
(10) Pub. No.: US 2012/0253143 A1
(43) Pub. Date: Oct. 4, 2012

(54) METHOD OF MEASURING A SKIN AGENT TRANSFERRED TO SKIN

(76) Inventor: Raphael Warren, Amberly Village, OH (US)

(21) Appl. No.: 13/079,444

(22) Filed: Apr. 4, 2011

(51) Int. Cl. A61B 5/00

(52) U.S. Cl. 600/306

(57) ABSTRACT

A method for measuring an amount of a skin agent transferred from an absorbent structure to an area of skin. An absorbent structure that includes a skin agent is provided and is applied to an area of skin. A portion of the skin agent transfers from the absorbent article to the area of skin. The absorbent article is removed and an extracting medium comprising an extraction component that does not irritate the skin is placed in the area of skin from which the absorbent structure was removed. The extracting medium is removed and the amount of skin agent extracted by the extracting medium is measured.

### FIELD OF THE INVENTION

The present invention relates to methods for determining the amount of a skin agent transferred from an absorbent structure to human skin.

### BACKGROUND OF THE INVENTION

Manufacturers of absorbent structures have recognized that various chemical skin agents can be added to absorbent structures either to treat a negative skin condition such as irritation, disease or infection, or to provide a beneficial effect such as a pleasing odor or lubrication. Absorbent structures such as diapers and feminine hygiene articles are normally worn in close and sustained contact with an area of skin and/or hair of a user and located in areas of the body where there is frequent motion. This motion can lead to a macerated skin condition through the friction created by the movement of the absorbent structure across the surface of an area of skin, leading to considerable discomfort. Additionally, these semi-occluded areas of the body are generally hydrated and contain microorganisms and proteases, which can further compromise the skin and promote feelings of discomfort, the feel of skin chafing, and malodor. To try and prevent these conditions, chemical treatments such as lotion compositions have been added to absorbent structures to soothe the feel of irritation and to lubricate and reduce the friction between the surface of the absorbent structure and the surface of an area of skin. Additionally, absorbent structures have used other skin agents to provide beneficial effects to users such as vitamins, protease inhibitors, and/or anti-microbial treatments.

Manufacturers also recognize that to produce a longer and more sustained effect from the chemical skin agent it should not only be transferred from the absorbent structure to the surface of the affected area of skin, but also that the chemical skin agent should at least partially be absorbed by an area of skin. The absorption of the skin agent by the skin allows the skin agent to provide a desired effect, without it having the risk of being reabsorbed by the absorbent structure, surface evaporation, or being moved to another area of the body by the movements of a user.

One common method of measuring the amount of skin agent transferred to an area of skin uses an adhesive tape to measure the amount of a skin agent transferred from an absorbent structure to an area of skin. The adhesive layer of the tape is placed on an area of skin directly opposite to where an absorbent structure will be worn and will have contact. During the wear time of the absorbent structure, the skin agent is transferred to the tape surface facing the absorbent structure. The tape is removed after a predetermined amount of time and analyzed to measure the amount of skin agent transferred to the tape surface.

There are several problems with using this method in a woman's vaginal area. For example, the tape is generally a smooth surface compared to the irregular topography of skin, which reduces its surface area as compared to an equally sized area of skin. In addition to the topographical surface differences, there are biochemical and thermodynamic differences in the vaginal area versus other regions of the body that may impact the tape stripping results.

Methods of measuring the amount of skin agent absorbed by the skin include tape stripping or cryanolate glue. Both of these methods can be unsuitable for use in the vaginal area due to vulvar sensitivity and the properties of the skin and hair in that region. As such, it would be desirable to provide a method that could measure the amount of a skin agent transferred from an absorbent structure to an area of skin. Additionally, it would be desirable to provide a method that could measure the amount of skin agent transferred from an absorbent structure that was absorbed by an area of skin.

### SUMMARY OF THE INVENTION

A method is provided for measuring an amount of a skin agent transferred from an absorbent structure to an area of skin. The method includes providing an absorbent structure, wherein the absorbent structure comprises a skin agent; applying the absorbent structure to an area of skin; transferring from the absorbent structure to the area of skin at least a portion of the skin agent; removing the absorbent structure from the area of skin; providing a extracting medium, wherein the extracting medium comprises an extraction component; placing the extracting medium in contact with the skin; removing the extracting medium from contact with the skin; and measuring an amount of skin agent in the extracting medium.

A method is provided for measuring an amount of a skin agent transferred from an absorbent article to an area of skin in the vulvar region. The method includes providing an absorbent article, wherein the absorbent article comprises a skin agent; applying the absorbent article to an area of skin, transferring from the absorbent article to the area of skin at least a portion of the skin agent; removing the absorbent article from the area of skin; providing an extracting medium, wherein the extracting medium comprises an extraction component; placing the extracting medium in contact to the area of skin from which the absorbent article was removed; removing the extracting medium from the area of skin; optionally repeating the steps of providing a extracting medium, wherein the extracting medium comprises an extraction component; placing the extracting medium in contact with the area of skin from which the absorbent article was removed; removing the extracting medium from the area of skin; and measuring an amount of skin agent in the extracting medium.

A method is provided for measuring an amount of skin agent from an area of skin in the vulvar region. The method includes providing an extracting medium, wherein the extracting medium consists of ethanol and cyclohexane; placing the extracting medium in contact with the area of skin from which an absorbent article comprising a skin agent was removed; removing the extracting medium from the area of skin; optionally repeating the steps of providing the extracting medium consisting of ethanol and cyclohexane; placing the extracting medium in contact with the area of skin from which the absorbent article comprising the skin agent was removed; removing the extracting medium from the area of skin; and measuring an amount of the skin agent in the extracting medium.

### BRIEF DESCRIPTION OF THE DRAWINGS

FIG. 1 is an erythema grading scale ranging from 0 to 4.

### DETAILED DESCRIPTION OF THE INVENTION

It has been discovered that data concerning the amount of a skin agent transferred from an absorbent structure such as a feminine hygiene pad or diaper to an area of (human) skin, can be gathered using methods described herein which can comprise various steps. A wide variety of data on the transfer of a skin agent from an absorbent structure to an area of skin can be obtained in a cost effective manner while still providing reproducible results. In addition, the methods both can generate data used to quantify the amount of a skin agent transferred from an absorbent structure to an area of skin, and may also quantify the amount of skin agent absorbed by an area of skin.

The inventive methods can be used to determine if skin agents present on or in an area of skin will positively or negatively affect the transfer or absorption of other skin agents from an absorbent structure to the area of skin. The inventive methods can be used to measure the amount of skin agent transferred to or absorbed by an area of skin after the removal of the absorbent structure. For example, the inventive methods can be used to determine how long a skin agent remains on or in the area of skin after removal of the absorbent structure. In addition, after removal of the absorbent structure, an area of skin to which an absorbent structure has been applied can be further treated, for example, by washing, wiping or scrubbing to determine the effect, if any, upon the skin agent.

As used herein, the term “absorbent article” refers to devices which absorb and contain body exudates, and, more specifically, refers to devices which are placed against or in proximity to the body of the wearer to absorb and contain the various exudates discharged from the body. The term “absorbent articles” includes sanitary napkins, panty liners (or pantiliners), incontinence devices, interlabial products, tampons, diapers, and training pants.

“Absorbent structure”, as used herein, refers to a structure which comprises one or more natural fibers, synthetic fibers or materials, foams such as for example those formed from High Internal Phase Emulsions (HIPE) or combinations thereof. As used herein “absorbent structure materials” refers to materials that are used to produce, result from the production of or form at least a part of an absorbent structure and which may be transferred to an area of skin. The term “absorbent structure” includes “absorbent articles.”

As used herein, “area of skin” refers to an area of skin on the body of a test subject that may have absorbent structures applied thereto.

As used herein, the term “botanical agents” refers to the chemically active components, of various plants and plant substances. Botanical agents can include any water-soluble or oil-soluble chemically active component extracted from a particular plant. Examples of extracted botanical agents are chemically active components extracted from echinacea, yucca glauca, willow herb, basil leaves, aloe, oregano, carrot root, grapefruit fruit, fennel, rosemary, thyme, blueberry, bell pepper, blackberry, blackcurrant fruit, tea leaves, coffee seed, dandelion root, date palm fruit, gingko leaf, hawthorn berries, licorice, sage, strawberry, sweet pea, tomato, vanilla fruit, absinthe, arnica, centella asiatica, chamomile, comfrey, cornflower, horse chestnut, ivy (Herdera helix), magnolia, mimosa, oat extract, pansey, scullcap, seabuckthorn, white nettle, witch hazel and any combinations thereof.

“Emollients”, as used herein, refers to materials that soften, soothe, supple, coat, lubricate, moisturize, or cleanse the area of skin.

As used herein, “ester-functional quaternary ammonium compounds” refers to compounds including but not limited to at least one of diester dialkyl dimethyl ammonium salts such as diester ditallow dimethyl ammonium chloride, monoester ditallow dimethyl ammonium chloride, diester ditallow dimethyl ammonium methyl sulfate, diester di(hydrogenated)tallow dimethyl ammonium methyl sulfate, diester di(hydrogenated)tallow dimethyl ammonium chloride, or mixtures thereof. In one embodiment, the surface treating composition comprises diester ditallow dimethyl ammonium chloride and/or diester di(hydrogenated) tallow dimethyl ammonium chloride, both commercially available from Witco Chemical Company Inc. of Dublin, Ohio under the tradename “ADOGEN SDMC™”.

As used herein, “immobilizing agent” refers to a component of the lotion that acts to prevent migration of the emollient so that it can remain primarily on the surface of the absorbent structure to which it is applied so that it may deliver maximum softening benefits as well as be available for transferability to the area of skin.

As used herein, “inorganic metal oxides” may be natural or synthetic and generally fall within two groups: photoactive and non-photoactive nanotechnology agents. General examples of photoactive metal oxide nanotechnology agents include zinc oxide and titanium oxide. Photoactive metal oxide nanotechnology agents require photoactivation from either visible light (e.g. zinc oxide) or from UV light (e.g. titanium oxide). Zinc oxide coatings have generally been used as anti-microbial agents or as anti-fouling agents.

As used herein, “inorganic nanotechnology agents” may include but are not limited to oxides such as inorganic metal oxides, silicates such as layered clay minerals, carbonates and hydroxides.

As used herein, “layered clay minerals” are clays that may be either naturally occurring or synthetic and include those in the geological classes of smectites, kaolins, illites, chlorites, attapulgites and mixed layer clays. Variants and isomorphic substitutions of these layered clay minerals offer unique applications. Smectites, include: montmorillonite, bentonite, pyrophyllite, hectorite, saponite, sauconite, nontronite, talc, beidellite, volchonskoite and vermiculite. Kaolins include: kaolinite, dickite, nacrite, antigorite, anauxite, halloysite, indellite and chrysotile. Illites include: bravaisite, muscovite, paragonite, phlogopite and biotite. Chlorites include: corrensite, penninite, donbassite, sudoite, pennine and clinochlore. Attapulgites include: sepiolite and polygorskyte. Mixed layer clays include: allevardite and vermiculitebiotite.

As used herein, “lotions” may comprise emollients and/or immobilizing agents and may be in the form of emulsions or dispersions. Lotions may include solids, gel structures, polymeric material, a multiplicity of phases (such as oily and water phases) and emulsified components. Lotions may be shear thinning or they may strongly change their viscosity around skin temperature to allow for transfer and easy spreading on an area of skin. Lotions may be semi-solid or of high viscosity so they do not substantially flow without activation during the life of the product or gel structures. Lotions may sooth, moisturize, and/or lubricate the area of skin.

As used herein, “nanotechnology agents” are organic or inorganic nanotechnology agents having average diameters of about 500 μm or less. Particle size distributions of the nanotechnology agents may fall anywhere within the range from about 2 μm to less than about 500 μm, alternatively from about 2 μm to less than about 100 μm, and alternatively from about 2 μm to less than about 50 μm Nanotechnology agents can also include crystalline or amorphous materials.

As used herein, “non-absorbent structure” refers to a structure that comprises one or more films.

As used herein, “non-photoactive metal oxide nanotechnology agents” do not use UV or visible light to produce the desired effects. Examples of non-photoactive metal oxide nanotechnology agents include, but are not limited to silica and alumina nanotechnology agents, and mixed metal oxide nanotechnology agents including, but not limited to saponites, and hydrotalcite. Aluminum can be found in many naturally occurring sources, such as kaolinite and bauxite.

As used herein, “pharmaceutical agents” refer to drugs for treating diseases locally or systemically, nutrients or other biologically active compounds or herbal extracts, and minerals to improve general health or local skin/mucous tissue conditions. Pharmaceutical agents include, but are not limited to, any material capable of exerting a biological effect on a human body, such as therapeutic drugs, including, but not limited to, organic and macromolecular compounds such as polypeptides, proteins, amino acids, and nucleic acid materials comprising DNA (deoxyribose nucleic acid) or RNA (ribonucleic acid), and nutrients.

As used herein, “quaternary ammonium compounds” are those compounds that may be used in the present invention including but not limited to at least one of dialkyldimethylammonium salts such as ditallowdimethylammonium chloride, ditallowdimethylammonium methylsulfate, di(hydrogenated tallow) dimethylammonium chloride or mixtures thereof. In one example, the surface treating composition comprises di(hydrogenated tallow) dimethylammonium chloride, commercially available from Witco Chemical Company Inc. of Dublin, Ohio as Varisoft 137®.

As used herein, “skin agent” refers to a biological or chemical composition that may be part of, present in, or on the surface of an absorbent structure. Skin agents may be capable of preventing, reducing, and/or eliminating skin disorders, particularly skin disorders associated with erythema, malodor, and bacterial infections.

As used herein, “skin healants” refer to components used to heal the skin. Skin healants include, but are not limited to, at least one of zinc oxide, vitamins, such as vitamin B3, vitamin E, sucrose esters of fatty acids, anti-inflammatory compounds, lipids, inorganic anions, inorganic cations, protease inhibitors, sequestration agents, alpha bisalbolol, or mixtures thereof.

As used herein, “extracting medium” refers to a liquid containing a carrier and a solvent, the solvent being capable of extracting lipids from the skin.

As used herein, “surface treating compositions” refer to compositions that may increase or decrease the surface friction of the surface of an absorbent structure. In certain embodiments, the surface treating composition will reduce the surface friction of the surface of the absorbent structure compared to a surface of the absorbent structure without such surface treating composition.

As used herein, “volumetric unit” refers to a container that has a set three-dimensional space a substance (solid, liquid, gas, or plasma) may occupy.

As used herein, “vulvar or vaginal” refers to the vulva, the female external genitalia including the labia majora, labia minora, clitoris, the tiny glands called Bartolin's glands, and the entrance to the vagina (the vestibule of the vagina).

The methods described herein can have a wide application for the measurement of the transfer and absorption by an area of skin of skin agents from a variety of absorbent structures and can utilize a variety of test subjects.

The method for measuring an amount of skin agent transferred from an absorbent structure to an area of skin can include providing an absorbent structure, wherein the absorbent structure comprises a skin agent; applying the absorbent structure to an area of skin; transferring from the absorbent structure to the area of skin at least a portion of the skin agent; removing the absorbent structure from the area of skin; providing an extracting medium, wherein the extracting medium comprises an extraction component; placing the extracting medium in contact with the area of skin from which the absorbent structure was removed; removing the extracting medium from the area of skin; optionally repeating the prior three steps; and measuring an amount of skin agent in the extracting medium.

The measurement of the transfer of skin agent from an absorbent structure to an area of skin of a test subject can be initiated by applying an absorbent structure to the area of skin. Alternatively, the transfer of a skin agent may occur by applying a non-absorbent structure to an area of skin of a test subject or may occur by applying a skin agent in direct contact with an area of skin of a test subject.

Any suitable area of skin of the human body can be used. The area of skin may have any surface contour such as planar, rounded or irregular. The area of skin may have hair or no hair, for example a shaved area of skin. There can be more than one area of skin on a test subject. The area of skin may be in the vulvar region. The “vulvar region” is understood to include the region from the posterior gluteal groove and perineum to the anterior mons pubis and laterally between the intertriginous zones and may include the intertriginous zones. In other embodiments, the area of skin may be mucosal skin. The area of skin may be axilla, popliteal fossa, urogenital area, talus, buccala, auris auricula, palpebra, buccocervical, digitus web, or crapus, cubitus.

The skin area may be of any type or size. For example, the area of skin may have a size of about 3 cm×3 cm. In certain other embodiments the area may be about 6 cm×4 cm. In certain other embodiments the area may be about 2.5 cm x 2.5 cm. In certain other embodiments the area may be circular having an area of at least about 0.5 cm², preferably at least about 1 cm², more preferably at least about 3 cm². In certain embodiments, Tegaderm™, or transparent medical dressing, is placed around the skin area. Additionally, the area may be any suitable shape such as square, rectangular, oval, and circular or any other desired shape. The type, size and/or shape of the area may be determined by factors such as the location of skin the extracting medium will be applied to, the test subject, the length of time an absorbent structure is applied to an area of skin, the analytical sensitivity to the skin agent, the absorbent structure and/or the type of skin agent being measured. For example, if the absorbent structure has only minute quantities of a skin agent or if it is known or estimated that the skin agent will not apply well to an area of skin, a larger area of skin may be tested (to collect more of the skin agent).

More than one absorbent structure may be applied to an area of skin before application of the extracting medium to the area of skin. The absorbent structures may be applied to an area of skin either concurrently or consecutively. The absorbent structures applied to an area of skin may differ, for example, two different types of the same absorbent structures may be applied to an area of skin, such as a first type of feminine hygiene pad followed by a second type of feminine hygiene pad, or the absorbent structures could differ such as a diaper applied to an area of skin followed by a wipe.

After a predetermined period of time, the absorbent structure is removed from an area of skin by either the test subject or a technician. The absorbent structure may be applied to an area of skin for any suitable period of time, such as, for example, a period of time that corresponds to that absorbent structure's typical application time during normal use. For example, test subjects could be instructed to change their feminine hygiene menstrual pads about every three hours to correspond with the typical application time of feminine hygiene menstrual pads or to change their feminine hygiene pantiliners about every eight hours. Alternatively, the absorbent structure may be applied to an area of skin for an extended period of time as compared to a typical application time, for example, to determine what effects if any there are on the area of skin, such as, for example, to determine if skin agent still being transferred from an absorbent structure to the area of skin after the typical application time or to determine if the transfer from an absorbent structure of the skin agent after the typical application time provides any detriment or benefit. The absorbent structure also may be applied to an area of skin for a shorter period of time as compared to a typical application time, for instance, to determine if a skin agent is transferred in a sufficient amount to the area of skin during a shorter application time.

Skin agents transferred from an absorbent structure can remain on the surface of or be absorbed by an area of skin. The absorbent structure may be removed using any suitable means such as hands, gloved hands, or forceps. Additionally, if there are multiple absorbent structures applied to one or more areas of skin on the body of a test subject, they do not all have to be removed at or around the same time. For example, all of the applied absorbent structures do not need to be removed from an area of skin at or around the same time as some of the absorbent structures may be left applied to the areas of skin while others are removed or even new absorbent structures are applied.

Following the removal of an absorbent structure from an area of skin, an extracting medium may be placed in contact with the skin. One or more isolated areas of skin may be placed in contact with the extracting medium. The extracting medium may be contained by any volumetric shape that may contain the necessary amount of extracting medium in contact with the target skin; however, the container should be designed to minimize leakage of the extracting medium outside the container. An example of a container can be a glass cylinder having an open area of approximately 3 cm². To place the extracting medium in contact with the skin, manipulation of the test subject and test site may occur, such as, for example, by positioning the skin site as close as parallel to the floor as possible. In certain embodiments, manipulation of test subjects can involve a dental examination chair or a gynecological chair.

The extracting medium may be transferred into a container that is positioned on the skin. Once placed in contact to an area of skin, the extracting medium may be agitated with an instrument such as a glass rod. The amount of extracting medium transferred into a container may be related to the area to be extracted. The total amount of extracting medium transferred to the container may be from about 0.5 ml to 50 ml, from about 1 ml to 30 ml, or from about 2 ml to 10 ml. Any suitable container for containing the extracting medium can be used, such as, for example, a container having an open area of 3 cm². The duration and level of agitation can be about 10 ml for less than about 5 min, about 7 ml for less than about 2 minutes, or about 5 ml for less than about one minute. In addition, the procedure may be repeated for a total of 5 times, using up to about 50 ml of extracting medium or a total of 3 times, using up to about 30 ml of extracting medium for a duration of up to about 15 min. Generally, there should be minimal to no contact with the skin by the agitator during the agitation of the extracting medium. In addition, when using containers having an open diameter of 0.5 cm², the duration and level of agitation can be about 2 ml for less than about 5 min, about 1 ml for less than about 2 minutes, or about 0.5 ml for less than about one minute.

After a predetermined length of time, the extracting medium may be removed from the area of skin in any suitable manner, such as, for example, a pipette. The length of time an extracting medium is in contact to an area of skin may depend on the area of skin tested, the extracting medium chosen, the skin agent (amount in absorbent structure, known or estimated skin absorbency), number of cycles chosen for extraction by the extracting medium, and/or area of skin (presence of hair, location on body, thickness of skin) or any other factors known in the art. For example, the length of time the extracting medium is placed in contact to an area of skin may be about 25 minutes or less, about 10 minutes or less, or about three minutes or less, and in certain other embodiments the length of time the extracting medium is placed in contact to an area of skin may be about one minute or less. In certain addition, the extracting medium may be agitated without contacting the skin. This may affect the amount of time the extracting medium is in contact with the skin. The extracting medium may be removed using a suitable device, such as, for example, a pipette or similar liquid transfer device. When the extracting medium is removed, it may be stored in a container such as a sample container glass jar with closure available from VWR Scientific, West Chester, Pa. until analysis. Individual extractions of the extracting medium can be stored separately for testing accuracy. The extracted samples can be dried down under a stream of nitrogen, if desired, and stored at −70° C. for up to 6 months.

The placement in contact and later extraction of the extracting medium from the area of skin of a test subject comprises one cycle. The total number of cycles may vary. In certain embodiments, the number of cycles may be determined by the skin agent on the surface of the area of skin and the amount of skin agent absorbed by the area of skin. The amount of skin agent absorbed by the area of skin may be influenced by factors such as where the area of skin is located on the body (presence of hair, thickness of area of skin, keratinization of the area of skin i.e., palms or soles will be different from the vulvar or back areas), presence of any other substances on the surface of the area of skin such as sebum that may act as barriers to absorption, or the oil/water partition coefficient property of the skin agent (i.e., hydrophobic agents tend to be absorbed by an area of skin more readily than hydrophilic agents), or the molecular weight of the skin agent (i.e., smaller molecular weight ingredients tend to be absorbed by an area of skin more readily than high molecular weight agents). In certain embodiments, the total number of cycles is from 1 to about 5.

Factors may be used to determine the number of cycles that will remove skin agent from the surface of the area of skin, such as the area of skin (presence of hair, smooth surface as in the volar forearm or rougher surface as in the face, age of test subject), skin agent, and/or length of time an absorbent structure was applied to an area of skin. For example, a technician may make a visual determination as to the presence of a skin agent on the area of skin surface.

Where there is more than one cycle, to help ensure that successive extraction by extracting medium are being done to the same area of skin, the approximate edges of the skin area tested can be marked on the area of skin by using a suitable skin marking instrument. After removal of the extracting medium, the marks are left on the area of skin so that subsequent extracting medium may be applied to the same area of skin.

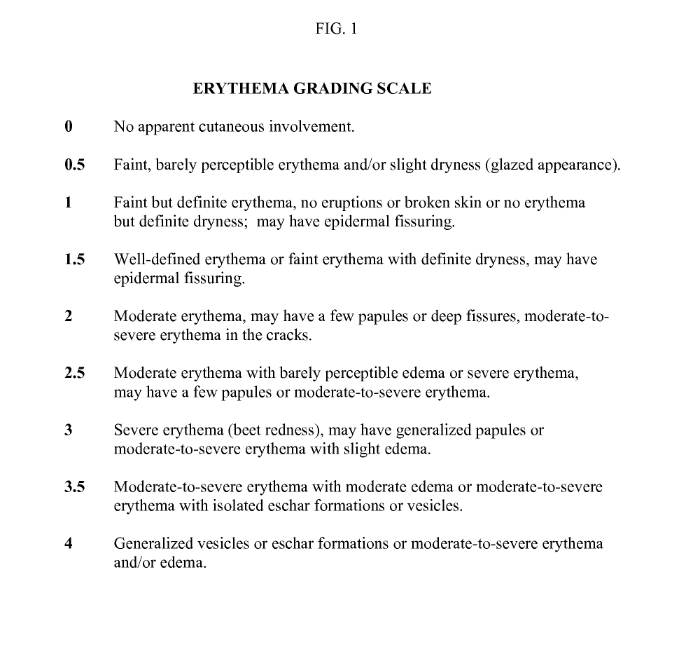

After the extracting medium is removed, the skin area is viewed and an erythema grade is assigned and recorded. The erythema grade is based on a scale ranging from 0 to 4 as shown in FIG. 1. A 0 represents “no apparent cutaneous involvement” while a 4 represents “generalized vesicles or eschar formations or moderate to severe erythema and/or edema.”

After the extracting medium is removed from contact with the area of skin, the amount of skin agent extracted is measured using suitable analytical methods. Any appropriate method of analysis can be used based on factors such as the skin agent, absorbent structure, or wearer types, such as, for example, as described herein.

To help ensure that a proper group of human test subjects are included in a test using a method of the present invention measuring the amount of skin agent transferred from an absorbent structure to an area of skin, potential test subjects can be pre-screened prior to commencement of a test. Depending on the absorbent structure and the skin agent being tested for, several selection criteria for test subjects can be used to select for an appropriate group of test subjects, for example: according to sex, age, sensitivities, sickness or maladies, and having a test area free of cuts, lesions, and sunburn. There can be more than one group and the size of the group can vary. A test subject in a group may have the same or different absorbent structures applied and/or the same or different skin agent amounts measured from an area of skin as compared to other test subjects within the same group. The size of the group may be determined by the absorbent structure and/or the skin agent being measured. Test subjects may be selected according to inclusion and exclusion criteria, before being tested by the methods of the present invention. The selection criteria can be administered to the test subjects by any method known in the art, for example as a form to be completed by a test subject or as a questionnaire administered by a technician. Suitable inclusion criteria can include: test subject (1) is at least eighteen years old; (2) is in general good health; (3) is willing to refrain from using lotions, creams, powders or other skin preparations on the area of skin during their participation in the study; (4) is willing to have the hair on the area of skin removed, or trimmed, or shortened, preferably shortened; (5) is willing to refrain from shaving the area of skin during their participation in the study; (6) is willing to refrain from swimming/hot tub use during their participation in the study; (7) is willing to refrain from tanning/sun exposure to the test area during their participation in the study; (8) agrees to refrain from sexual activity during their participation in the study if the area of skin is in the vulvar, labial, or general vaginal area including the upper inner thigh area.

Suitable exclusion criteria can include: test subject (1) has sunburn, acne, abrasions, scar tissue, tattoos, or any other skin abnormality at the area of skin; (2) has psoriasis, eczema, skin cancer, or any active dermatitis at the area of skin; (3) is using oral or topical corticosteroids; (4) has a known irritancy or discomfort in the area of skin which would prevent test subject from wearing an absorbent structure; (5) is currently pregnant; (6) has diabetes; (7) has kidney disease; (8) has heart and/or circulatory disease, including blood clots; (9) has varicosities which would interfere with a test subject wearing an absorbent structure during their participation in the study; and (10) has arthritis in the lower extremities.

The skin agent can be any suitable agent, including, for example, lotions, surface treating compositions, nanotechnology agents, encapsulated time release agents, skin healants, anesthetics, analgesics, perfumes, such as long lasting or enduring perfumes, antibacterial agents, antiviral agents, botanical agents, disinfectants, pharmaceutical agents, film formers, dyes, inks, colorants, surfactants, absorbents, wet strength agents, deodorants, opacifiers, astringents, solvents, biological agents such as bacteria, viruses and their toxins, absorbent structure materials or mixtures thereof.

Emollients can be any suitable agent, including, for example, glycols (such as propylene glycol and/or glycerine), polyglycols (such as triethylene glycol), petroleum-based materials, fatty acids, fatty alcohols, fatty alcohol ethoxylates, fatty alcohol esters and fatty alcohol ethers, fatty acid ethoxylates, fatty acid amides and fatty acid esters, alkyl ethoxylates, hydrocarbon oils (such as mineral oil), squalane, fluorinated emollients, silicone oil (such as dimethicone) or mixtures thereof.

Petroleum-based emollients include 16 to 32 carbon atom hydrocarbons, or mixtures of 16 to 32 carbon atom hydrocarbons. Petroleum based hydrocarbons having these chain lengths include petrolatum (also known as “mineral wax,” “petroleum jelly” and “mineral jelly”). Petrolatum usually refers to more viscous mixtures of hydrocarbons having from 16 to 32 carbon atoms. A petrolatum that may be used is available from Witco, Corp., Greenwich, Conn. as White Protopet® 1 S.

Fatty acid ester emollients that may be used include but are not limited to those derived from long chain C₁₂-C₂₈ fatty acids, such as C₁₆-C₂₂ saturated fatty acids, and short chain C₁-C₈ monohydric alcohols, such as C₁-C₃ monohydric alcohols. Nonlimiting examples of fatty acid ester emollients include but are not limited to at least one of methyl palmitate, methyl stearate, isopropyl laurate, isopropyl myristate, isopropyl palmitate, ethylhexyl palmitate, or mixtures thereof. Fatty acid ester emollients can also be derived from esters of longer chain fatty alcohols (C₁₂-C₂₈, such as C₁₂-C₁₆) and shorter chain fatty acids e.g., lactic acid, such as lauryl lactate and cetyl lactate.

Alkyl ethoxylate type emollients may include but are not limited to at least one of C₁₂-C₁₈ fatty alcohol ethoxylates having an average of from 3 to 30 oxyethylene units, such as from about 4 to about 23. Nonlimiting examples of such alkyl ethoxylates include laureth-3 (a lauryl ethoxylate having an average of 3 oxyethylene units), laureth-23 (a lauryl ethoxylate having an average of 23 oxyethylene units), ceteth-10 (acetyl ethoxylate having an average of 10 oxyethylene units), steareth-2 (a stearyl ethoxylate having an average of 2 oxyethylene units), steareth-10 (a stearyl ethoxylate having an average of 10 oxyethylene units) or mixtures thereof. These alkyl ethoxylate emollients may be used in combination with the petroleum-based emollients, such as petrolatum, at a weight ratio of alkyl ethoxylate emollient to petroleum-based emollient of from about 1:1 to about 1:3 in certain embodiments, from about 1:1.5 to about 1:2.5 in certain other embodiments.

Suitable oils include but are not limited to at least one of mineral oil, silicone oil, silicone gels or mixtures thereof. Silicones include but are not limited to at least one of polydimethylsiloxanes, aminosilicones, cationic silicones, quaternary silicones, silicone betaines or mixtures thereof. In certain embodiments, the siloxane may be an aminofunctional polydimethylsiloxane such as aminoethylaminopropyl polydimethylsiloxane.

Suitable oils further include natural fats and oils containing omega fatty acids. These can include oleic canola oil (Brassica campestris, B. napus, B. rapa; characterized by having an oleic content greater than 70%, e.g., hi oleic canola oil, very high oleic canola oil, or partially hydrogenated canola oil), manila kernel oil (Sclerocarya birrea), palm oil (Elaeis Guineensis Oil), palm olein, palm stearin, palm superolein, pecan oil, pumpkin seed oil, oleic safflower oil (Carthamus Tinctorius; characterized by having an oleic content of greater than about 30% and omega-6 fatty acid content of less than about 50%, e.g., hi oleic safflower oil), sesame oil (Sesamum indicum, S. oreintale), soybean oil (Glycine max, e.g., hi oleic soybean, low linolenic soybean oil, partially hydrogenated), oleic sunflower oil (Helianthus annus; characterized by having an oleic content of greater than about 40%, e.g., mid oleic sunflower or high oleic sunflower oil), and mixtures thereof. Oleic canola oil, palm oil, sesame oil, hi oleic safflower oil, hi oleic soybean oil, mid oleic sunflower oil, and high oleic sunflower oil are common plant-bred derived oils and may be also be derived from non-genetically modified organisms (non-GMO). Non-limiting examples of oil materials are commercially-available from a number of vendors, including Cargill for partially hydrogenated soybean oil (i.e., Preference® 110W Soybean Oil or Preference® 300 Hi Stability Soybean Oil), mid oleic sunflower oil (i.e., NuSun® Mid-Oleic Sunflower Oil), high oleic sunflower oil (i.e., Clear Valley® High Oleic Sunflower Oil), high oleic canola oil, very high oleic canola, and partially hydrogenated low erucic rapeseed oil (i.e., Clear Valley® 65 High Oleic Canola Oil and Clear Valley® 75 High Oleic Canola Oil); Lambert Technology for high oleic canola oil (i.e., Oleocal C104); Arch Personal Care for manila kernel oil; Pioneer for high oleic soybean oil (i.e., Plenish®); Asoyia for low linolenic soybean oil (i.e., Ultra Low Linolenic Soybean Oil®); and Dipasa, Inc. for refined sesame oil.

The natural fat and oil material can further comprise a blend of oils, including those described supra, as well as additional oil materials. Suitable additional oil materials can include acai berry oil, almond oil, avocado oil, beech oil, brazil nut oil, camelina sativa oil (family Brassicaceae, e.g. Camelina Sativa, Gold of Pleasure, False Flax, etc.), camellia seed oil, canola oil, carrot seed oil, cashew nut oil, caster oil, cherry kernel oil, chia oil, corn oil, cottonseed oil, hydrogenated cottonseed oil, evening primrose oil, filbert (hazelnut) oil, grapeseed oil, hemp oil, hickory nut oil, jojoba oil, kukui oil, lanolin, olive oil (Olea europaea), macadamia oil, maringa oil, meadowfoam oil, neem oil, palm kernel oil, olive oil, passionflower oil (family Passiflora, Passiflora Incarnata), peanut oil, peach kernel oil, pistachio nut oil, rapeseed oil, rice bran oil, rose hip oil, safflower oil, sorghum oil, soybean oil, sunflower seed oil, tall oil, vegetable oil, vegetable squalene, walnut oil, wheat germ oil, and mixtures thereof. The suitable oils can comprise from about 3% to about 50%, or from about 5% to about 40%, by weight of the oil material, of omega-6 fatty acid.

Immobilizing agents may include agents that prevent migration of the emollient into the absorbent structure such that the emollient remains primarily on the surface of the absorbent structure therefore facilitating the transfer of the lotion to area of skin. Immobilizing agents may function as viscosity increasing agents and/or gelling agents.

Immobilizing agents may include but are not limited to at least one of waxes such as ceresin wax, ozokerite, microcrystalline wax, petroleum waxes, fisher tropsch waxes, silicone waxes, paraffin waxes, polyethylene waxes, beeswax, fatty alcohols such as cetyl, cetaryl, cetearyl and/or stearyl alcohol, fatty acids and their salts such as metal salts of stearic acid, mono and polyhydroxy fatty acid esters, mono and polyhydroxy fatty acid amides, silica and silica derivatives, gelling agents, thickeners or mixtures thereof.

The surface treating composition may be a composition comprised of one or more surface treating agents that improves the tactile sensation of a surface of an absorbent structure as perceived by a user who holds the absorbent structure and rubs it across the area of skin. Such tactile perceivable softness can be characterized by, but is not limited to, friction, flexibility, and smoothness, as well as subjective descriptors, such as a feeling like lubricious, velvet, silk or flannel. The surface treating composition may or may not be transferable. In certain embodiments, the surface treating composition may be substantially non-transferable.

Examples of surface treating agents include but are not limited to at least one of polymers such as polyethylene and derivatives thereof, hydrocarbons, oils, silicones, siloxanes, organosilicones, quaternary ammonium compounds, ester-functional quaternary ammonium compounds, fluorocarbons, substituted C₁₀-C₂₂ alkanes, substituted C₁₀-C₂₂ alkenes, in certain embodiments, the substituted C₁₀-C₂₂ alkenes may be derivatives of fatty alcohols, polyols, derivatives of polyols such as esters and ethers, sugar derivatives such as ethers and esters or mixtures thereof.

In one embodiment, the surface treating composition can comprise a microemulsion and/or a macroemulsion of a surface treating agent in water. In such an example, the concentration of the surface treating agent within the surface treating composition may be from about 3% to about 60% and/or from about 4% to about 50% and/or from about 5% to about 40%. Nonlimiting examples of such microemulsions are commercially available from Wacker Chemie AG Munchen, Germany (MR1003, MR103, MR102). A nonlimiting example of such a macroemulsion is commercially available from General Electric Silicones, Wilton, Connecticut (CM849).

The absorbent structure may be any suitable absorbent structure. The absorbent structure may be embossed, pattern-densified, creped, uncreped, or include combinations thereof. The absorbent structure may also comprise a nonwoven web, cellulosic fiber containing web or combinations thereof. Nonlimiting examples of absorbent structures include absorbent articles such as feminine hygiene pads, interlabial pads, and pantiliners. Other nonlimiting examples of absorbent structures include diapers, training pants, and adult incontinence products. Other examples of absorbent structures include sanitary tissues, facial tissues, toilet tissues, paper towels, wipes such as cleaning wipes and dusting wipes, textiles, fabrics, cotton balls, swabs or pads, or wound dressings.

The absorbent structure materials may be any suitable material. For example, polymers or monomers used to produce foam for an absorbent structure may be absorbent structure materials or absorbent structure materials may be unreacted intermediates or processing aids used in the production of an absorbent structure such as foam, for example, emulsifiers or initiators.

The extracting medium may include a carrier and an extraction component. The extracting medium may comprise of any material that does not exceed skin erythema levels greater than 1.0, such as, for example, not greater than 0.5 after up to five extractions at the extraction site. The skin erythema grading scale chosen ranges from 0 to 4. See FIG. 1. A 0 is defined as “no apparent cutaneous involvement.” A 4 is defined as “generalized vesicles or eschar formations or moderate to severe erythema and/or edema.” A score of 0.5 is defined as “faint, barely perceptible erythema and/or slight dryness (glazed appearance).” A score of 1 is defined as “faint but definite erythema, no eruptions or broken skin or no erythema but definite dryness; may have epidermal fissuring.” Some representative carriers include methanol, ethanol, 1-propanol, 1-butanol, acetic acid, formamide, acetone, ethyl acetate, acetonitrile, hexane, and diethyl ether.

Suitable extraction components have a Log P or octanol/water partition coefficient equal to or greater than 1, such as, for example, cyclohexane, n heptane, 2 pentanol, 3 methyl 2 butanol, 1 hexanol, 1,8 cineole, 2,5 dimethylfuran, biphenyl, and methylcyclohexane. Other suitable extraction components include: cycloheptane, cyclooctane, trifluoromethane, 1 s bromohexane, 2 hexanol, 3 pentanol, 1 chloropropane, ethylene, and trans 1, 3 hexadiene. A preferred extracting medium is ethanol-cyclohexane, 8:2 v/v (USP grade absolute ethanol and 99.9+% HPLC grade cyclohexane).

### EXAMPLE 1

### Lipid Analysis

This example demonstrates analysis of skin lipids from the forearm, inner thigh, and labia majora.

For analyses, samples that had been dried down were reconstituted in chloroform: methanol (2:1), preferably to 20 mg/ml.

Analytical thin-layer chromatography was used to visualize and quantify the relative abundance of skin lipids. It was performed using 20×20 cm glass plates coated with a 0.25 mm layer of silica gel G (Adsorbasil Plus-1; Alltech Associates, Deerfield Ill.). The glass plates were rinsed with chloroform:methanol (2:1) and activated in an oven set to approximately 110° C. The silica plate was divided into approximately 6 mm wide lanes, and calibrated glass capillaries were used to apply approximately 5 μl samples 2-3 cm from the bottom edge of the plate. The chromatogram was developed two ways. For resolving non-polar lipids hexane was used to approximately 20 cm, followed with toluene to approximately the next 20 cm, followed with hexane:ethyl ether:acetic acid (70:30:1) to approximately the next 12 cm. To resolve the polar lipids, the plate was developed chloroform:methanol:water (40:10:1 v/v) to approximately 10 cm, followed by chloroform:methanol:acetic acid (190:9:1 v/v) to approximately the next 20 cm, followed by hexane:ethyl ether:acetic acid (70:30:1 v/v) to approximately the next 20 cm. The least polar lipids migrate to the top edge of the plate while the phospholipids are partially resolved on the lower one-third of the chromatogram. The chromatograms were air dried, sprayed with approximately 50% sulphuric acid, and slowly heated to approximately 220° C. on an aluminum slab on a hot plate to induce charring. Charred chromatograms were quantified from densitometer tracings (CS-9000; Shimadzu Corporation, Columbia Md.). After approximately 2 hrs, charring was generally complete, and the chromatogram were quantitated by photodensitometry. Lipids visualized can include ceramide EOS, ceramide NS, ceramide EOP/NP (these usually migrate together), ceramide AP, ceramide EOH, ceramide AS/NH (these usually migrate together), ceramide AH, cholesterol sulfate, cholesterol sulfate sphingosine complex, squalene, cholesterol ester, wax ester, triglyceride, fatty acid, and cholesterol. Standards used for identification of lipids as well as for quantitation included squalene, cholesteryl oleate, stearyl palmitate, tripalmitin, stearic acid, cholesterol (Sigma, St Louis Mo., USA), ceramide NP (Evonic Industries, Essen, Germany), cholesterol sulfate (synthetic). Standard curves were established using lipid amounts between 0.1 and 25 μg.

To determine the relative abundance of fatty acids esterified to ceramides (i.e., ceramide EOS or ceramide NS), the lipid extract was applied as thin streaks 2 cm from the bottom of the plate, and the chromatograms were developed for polar lipids as described above. After drying, the chromatograms were sprayed with an ethanolic solution of 2,7-dichlorofluorescein (Sigma Chemical Company, St. Louis Mo.), and lipid bands were visualized under UV light. The band corresponding to ceramide EOS was scraped from the plate, and the silica extract was placed in a small glass column. The ceramide was eluted with chloroform:methanol:water (50:50:1 v/v). After drying under nitrogen, the samples were treated with 10% boron trichloride (Aldrich, St Louis MO) in methanol at 50° C. for 1 hr. The reagent was evaporated under nitrogen. Fatty acid methyl ester (FAME) were isolated from each sample by preparative thin layer chromatography with a mobile phase of toluene, and samples were analyzed by gas-liquid chromatography using a 30 m EC-WAX quartz capillary column operated isothermally at 170° C. One instrument used was a Shimadzu GC-14A equipped with a flame ionization detector. Methylated fatty acid esters can include and not limited to palmitate (C16:0), palmitoleic (C16:1), stearate (C18:0), oleate (C18:1), linoleate (C18:2), linoleneate (C18:3), and arachidic (C20:0).

Alternatively, the relative abundance of fatty acids can be derivatized to FAMEs using a solution of BF₃ in methanol. The resulting methyl esters can be analyzed by gas chromatography and separated by chain length as well as degree of unsaturation using a polar capillary column with flame ionization detection. The methyl esters can be identified by comparison to a standard of known composition. The relative distribution of FAMEs can be based on normalized peak area percent using response factors generated from the standard of known composition.

TABLE 1
Polar Lipid Analysis (wgt %)

| | | (% Std | Inner | (% Std | Labia | (% Std |
|---|---|---|---|---|---|---|
| | Forearm | Dev) | Thigh | Dev) | Majora | Dev) |
| Ceramide EOS | 2.9 | 26.0% | 3.6 | 29.5% | 3.1 | 21.7% |
| Ceramide NS | 8.2 | 34.0% | 9.0 | 23.0% | 9.3 | 24.1% |
| Ceramide EOP | 10.5 | 36.1% | 13.9 | 35.2% | 11.6 | 33.2% |
| and Ceramide NP | | | | | | |
| Ceramide EOH | 3.4 | 18.3% | 3.4 | 56.3% | 3.4 | 61.6% |
| Ceramide AS and | 28.2 | 10.3% | 22.6 | 9.4% | 25.2 | 26.0% |
| Ceramide NH | | | | | | |
| Ceramide AP | 12.8 | 33.6% | 14.7 | 26.7% | 14.6 | 30.2% |
| Ceramide AH | 23.0 | 20.1% | 17.9 | 37.8% | 17.2 | 23.9% |
| Total Ceramide | 89.0 | 3.8% | 85.1 | 4.8% | 84.4 | 6.2% |
| Cholesterol | 5.0 | 35.8% | 6.8 | 16.8% | 7.5 | 38.7% |
| Sulfate | | | | | | |
| Sphingosine | | | | | | |
| Complex (CSSC) | | | | | | |
| Cholesterol | 6.0 | 32.3% | 8.2 | 37.6% | 8.1 | 31.1% |
| Sulfate (CS) | | | | | | |
| CSSC + CS | 11.0 | 30.9% | 14.9 | 27.5% | 15.6 | 33.3% |
| Total Polar Lipids | 32.7 | 29.3% | 34.8 | 25.5% | 36.7 | 24.2% |
| Recovered (μg | | | | | | |
| per 3.1 cm²) | | | | | | |

The amount of polar lipids collected was similar across all body sites (Table 1). Only marginal differences were observed for the relative abundance of ceramides, i.e., ceramide AH.

TABLE 2
Acylceramide Ester-linked Fatty Acid Derived from
Ceramide EOS (wgt %)

| | Forearm | Inner Thigh | Labia Majora |
|---|---|---|---|
| C16:0 | 31.6 | 40.2 | 36.7 |
| C16:1 | 3.4 | 5.1 | 3.4 |
| C18:0 | 20.2 | 19.9 | 19.8 |
| C18:1 | 19.1 | 19 | 20.9 |
| C18:2 (ω6) | 20.7 | 4.1 | 4.9 |
| C20:0 | 5 | 11.6 | 14.3 |

This data suggests a ˜75% reduction in the abundance of ω-6 linoleate from either the labia majora or the inner thigh relative to the forearm (Table 2). The ω-6 linoleate reduction appears to be compensated with an increase in the saturated fatty acids, palmitic and arachidic. An eight-fold reduction in linoleate from the palatal stratum corneum was compensated with an increase in palmitate and stearate.

Relative to the forearm, the labia and inner thigh were found to have a greater abundance of cholesterol sulfate sphingosine complex (CSSC) and cholesterol sulfate.

TABLE 3
Neutral Lipid Analysis (wgt %)

| | | (% Std | Inner | (% Std | Labia | (% Std |
|---|---|---|---|---|---|---|
| | Forearm | Dev) | Thigh | Dev) | Majora | Dev) |
| Squalene | 2.8 | 27.7% | 2.6 | 25.0% | 2.7 | 23.6% |
| Cholesterol Ester | 6.0 | 28.8% | 6.8 | 23.9% | 14.8 | 32.4% |
| Wax Ester | 13.3 | 20.7% | 14.9 | 20.4% | 11.3 | 26.9% |
| Triglycerides | 28.3 | 13.5% | 32.6 | 11.7% | 22.4 | 21.8% |
| Free Fatty Acids | 34.0 | 11.1% | 28.3 | 13.6% | 28.4 | 20.9% |
| Cholesterol | 15.5 | 22.5% | 14.8 | 12.8% | 20.4 | 26.3% |
| Total Neutral | 173.0 | 11.2% | 168.8 | 5.8% | 145.3 | 13.5% |
| Lipids Recoverd | | | | | | |
| (μg/3.1 cm2) | | | | | | |

The yield of neutral lipids from the labia majora was significantly less than either the forearm or the inner thigh (Table 3) and appeared to be contributed by a reduced yield of wax esters, triglycerides, and free fatty acids. In contrast the labia exhibited a greater abundance of cholesterol and cholesterol ester. These components are generally associated with keratnocyte/corneocyte breakdown.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm” All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

It will be understood that the embodiments described herein are merely exemplary, and that one skilled in the art may make variations and modifications without departing from the spirit and scope of the invention. All such variations and modifications are intended to be included within the scope of the invention as described hereinabove. Further, all embodiments disclosed are not necessarily in the alternative, as various embodiments of the invention may be combined to provide the desired result.

What is claimed is:

1. A method for measuring an amount of a skin agent transferred from an absorbent structure to an area of skin, the method comprising the steps of:
a. providing an absorbent structure, wherein the absorbent structure comprises a skin agent;
b. applying the absorbent structure to an area of skin;
c. transferring from the absorbent structure to the area of skin at least a portion of the skin agent;
d. removing the absorbent structure from the area of skin;
e. providing a extracting medium, wherein the extracting medium comprises an extraction component that does not irritate the skin;
f. placing the extracting medium in contact with a portion of the area of skin from which the absorbent structure was removed;
g. removing the extracting medium from the area of skin;
h. optionally repeating steps e, f and g; and
i. measuring an amount of the skin agent extracted by the extracting medium.

2. The method according to claim 1, wherein the absorbent structure is a feminine hygiene pad, interlabial pad, pantiliner, topsheet, diaper, training pant, adult incontinence product, sanitary tissue, facial tissue, toilet tissue, paper towel, wipe, textile, fabric, cotton ball, swab or pad, or wound dressing.

3. The method according to claim 1, wherein the extracting medium comprises a carrier and an extraction component.

4. The method according to claim 3, wherein the carrier is selected from the group consisting of methanol, ethanol, 1-propanol, 1-butanol, formic acid, acetic acid, formamide, acetone, methyl ethyl ketone (MEK), ethyl acetate, acetonitrile, N,N-dimethylformamide (DMF), diemthyl sulfoxide (DMSO), hexane, diethyl ether, methylene chloride, and combinations thereof.

5. The method according to claim 3, wherein the extraction component is selected from the group consisting of cyclohexane, n heptane, 2 pentanol, 3 methyl 2 butanol, 1 hexanol, 1, 8 cineole, 2, 5 dimethylfuran, biphenyl, methylcyclohexane, cycloheptane, cyclooctane, trifluoromethane, 1 s bromohexane, 2 hexanol, 3 pentanol, 1 chloropropane, ethylene, trans 1, 3 hexadiene, and combinations thereof.

6. The method according to claim 3, wherein the extraction component has a Log P equal to or greater than 1.

7. The method according to claim 1, wherein the skin agent comprises at least one of lotions, surface treating compositions, nanotechnology agents, encapsulated time release agents, skin healants, natural fats and oils, anesthetics, analgesics, perfumes, antibacterial agents, antiviral agents, botanical agents, disinfectants, pharmaceutical agents, film formers, dyes, inks, colorants, surfactants, absorbents, wet strength agents, deodorants, opacifiers, astringents, solvents, biological agents, absorbent structure materials or mixtures thereof.

8. The method according to claim 1, wherein the extracting medium is placed in contact with the skin using a volumetric unit.

9. The method according to claim 1, wherein the extracting medium is in contact with the skin for at least about 1 to at least about 5 minutes per extraction.

10. The method according to claim 1, wherein the volume of extracting medium placed in contact with the skin ranges from about 0.5 to 50 milliliters.

11. The method according to claim 1, wherein a component of skin agent measured is a lipid selected from the group consisting of ceramide EOS, ceramide NS, ceramide EOP/NP, ceramide AP, ceramide EOH, ceramide AS/NH, ceramide AH, cholesterol sulfate, cholesterol sulfate sphingosine complex, squalene, cholesterol ester, wax ester, triglyceride, fatty acid, cholesterol, and combinations thereof.

12. The method according to claim 1, wherein the component of skin agent measured is a fatty acid esterified to ceramide EOS or ceramide NS.

13. The method according to claim 1, wherein the area of skin comprises at least one of vulvar skin or mucosal skin.

14. A method for measuring an amount of a skin agent transferred from an absorbent article to an area of skin in the vulvar region, the method comprising the steps of:
a. providing an absorbent article comprising a topsheet, backsheet, and absorbent core, wherein the absorbent article comprises a skin agent;
b. applying the absorbent article to an area of skin;
c. transferring from the absorbent article to the area of skin at least a portion of the skin agent;
d. removing the absorbent article from the area of skin;
e. providing an extracting medium, wherein the extracting medium comprises an extraction component;
f. placing the extracting medium in contact with a portion of the area of skin from which the absorbent article was removed;
g. removing the extracting medium from the area of skin;
h. optionally repeating steps e, f and g; and
i. measuring an amount of the skin agent in the extracting medium.

15. The method according to claim 14, wherein the area of skin is at least one of axilla, popliteal fossa, urogenital area, talus, buccala, auris auricula, palpebra, buccocervical, digitus web, crapus, cubitus or combinations thereof.

16. The method according to claim 14, wherein the extraction component is selected from the group consisting of cyclohexane, n heptane, 2 pentanol, 3 methyl 2 butanol, 1 hexanol, 1, 8 cineole, 2, 5 dimethylfuran, biphenyl, methylcyclohexane, cycloheptane, cyclooctane, trifluoromethane, 1 s bromohexane, 2 hexanol, 3 pentanol, 1 chloropropane, ethylene, trans 1, 3 hexadiene, or any combinations thereof.

17. The method according to claim 14, wherein the skin agent comprises at least one of lotions, surface treating compositions, nanotechnology agents, encapsulated time release agents, skin healants, natural fats and oils, anesthetics, analgesics, perfumes, antibacterial agents, antiviral agents, botanical agents, disinfectants, pharmaceutical agents, film formers, dyes, inks, colorants, surfactants, absorbents, wet strength agents, deodorants, opacifiers, astringents, solvents, biological agents, absorbent structure materials or mixtures there.

18. A method for measuring an amount of a skin agent from an area of skin in the vulvar region, the method comprising the steps of:
a. providing an extracting medium consisting of ethanol and cyclohexane;
b. placing the extracting medium in contact with the area of skin from which an absorbent article comprising a skin agent was removed;
c. removing the extracting medium from the area of skin;
d. optionally repeating steps a, b and c; and
e. measuring an amount of the skin agent in the extracting medium.

19. The method according to claim 18, wherein the skin agent comprises at least one of lotions, surface treating compositions, nanotechnology agents, encapsulated time release agents, skin healants, and natural fats and oils.

20. The method according to claim 19, wherein the natural fats and oils contain an omega fatty acid content of at least about 3%, by weight of an oil material comprising an omega-6 fatty acid.
